(19) United States
(12) Patent Application Publication
(10) Pub. No.: US 2022/0203556 A1
(43) Pub. Date: Jun. 30, 2022

(54) SYSTEM FOR CONTROLLABLY ESTABLISHING ADHESION OR FRICTION WITH AN EXTERNAL BODY

(71) Applicant: THE GOVERNING COUNCIL OF THE UNIVERSITY OF TORONTO, Toronto (CA)

(72) Inventors: Benjamin David Hatton, Toronto (CA); Kurtis Allan Laqua, Welland (CA)

(21) Appl. No.: 17/605,571

(22) PCT Filed: Apr. 22, 2020
(86) PCT No.: PCT/CA2020/050532

§ 371 Date: Oct. 21, 2021

Related U.S. Application Data
(60) Provisional application No. 62/836,962, filed on Apr. 22, 2019.

(51) Int. Cl. B25J 15/00; A61B 34/00; B25J 15/08; A61B 90/00

(52) U.S. Cl. CPC B25J 15/0023; A61B 34/70; A61B 2090/064; B25J 15/08; A61B 90/06; B25J 15/0085

(57) ABSTRACT

A system for controllably establishing adhesion or friction with an external body. The system comprises a channel member and an interface member. The channel member comprises fluidic channels communicating positive or negative pressure. The interface member is disposed over the channel member. The interface member comprises exposed portions and topographic members. The exposed portions are flexible and exposed to the pressure communicated by the fluidic channels. The exposed portions deform under the pressure communicated by the fluidic channels and reform in the absence of the pressure communicated by the fluidic channels. The exposed portions assume a convex shaped under positive pressure and concave shaped configuration under negative pressure. The topographic members are provided over the exposed portions. The topographic members move based on the deformation of the exposed portions. The strength of adhesion varies based on the deformation of the exposed portions.

### BACKGROUND

Unless otherwise indicated herein, the materials described in this section are not prior art to the claims in this application and are not admitted to being prior art by inclusion in this section.

### FIELD

The subject matter in general relates to reversible adhesion. More particularly, but not exclusively, the subject matter is directed to configurable surfaces for reversible adhesion.

### DISCUSSION OF RELATED ART

Adhesion is the tendency of dissimilar particles or surfaces to mechanically cling or bond to one another and can refer to an interfacial adhesion strength in normal or shear loading. The adhesion mechanism can be divided into several types: chemical, electrostatic and mechanical, among others. Chemical adhesion is generally irreversible, whereas electrostatic or mechanical may be reversible adhesion.

In reversible electrostatic, magnetic or mechanical adhesion, the adhesion of one surface to another may be controllable. That is to say, the interfacial adhesion strength of the adhesive surface to another surface (known as the counterface) may be increased or decreased using electrostatic, magnetic or mechanical means, to adhere or let go. As an example, in a magnetically-induced adhesion, the surfaces may become adhered to each other by activating a magnetic field (for an electromagnet and a ferromagnetic surface), and the adhesion may be reduced by deactivating the magnetic field. On deactivation of the magnetic field, the surfaces may be separated from each other.

Smart materials are those that can change their properties due to an external control (such as external environment, or electrical signal). Therefore, a smart adhesion surface is a material that can actively change its interfacial adhesion strength to a countersurface. The effectiveness of a smart adhesion surface can be measured by: (1) interfacial adhesion strength (MPa) in normal or shear loading, (2) the switching ratio (SR)=F_{high}/F_{low}, where F_{high} is the adhesive force in the ‘on’ state, and F_{low} is that in the ‘off’ state, and (3) the switching time, the time required to switch from a high to low adhesive state.

The most widely used mechanical adhesion is the hook and the loop. In hook and loop adhesion, the hooks may curl around thread loops for adhesion and by providing an external mechanical force, the hooks may be unfastened from the loops. Smart adhesion materials have been designed to respond to temperature changes by altering the orientation or modulus of the hooks, providing an easier disengagement from the loops and controlling the interfacial adhesion strength. However, such a technology may be limited by the need for local temperature control, which may be generally difficult, impractical and with a high switching time.

Further, gecko-inspired dry adhesives may also be used as reversible adhesives. Gecko-inspired dry adhesives (Geckos) may use artificial setae or fibrils to adhere to external surfaces using van der Waals forces. However, gecko-inspired dry adhesives need a high density of setae, and require a physical peeling motion to reverse adhesion, which is not practical for many applications.

Another method for reversible adhesion may involve roughness or local curvature modification. Pneumatic actuation has been used to inflate a surface membrane and use the change in surface curvature and contact area (for dry adhesion) to grip and release objects. The switching ratio is generally low for these mechanisms (10-50).

In light of the foregoing discussion, there may be a need for an improved technique for mechanical reversible adhesion.

### SUMMARY

In one aspect, a system is provided for controllably establishing adhesion or friction with an external body. The system comprises a channel member and an interface member. The channel member comprises a plurality of fluidic channels communicating positive or negative pressure. The interface member is disposed over the channel member. The interface member comprises exposed portions and topographic members. The exposed portions are flexible and exposed to the pressure communicated by the fluidic channels. The exposed portions deform under the pressure communicated by the fluidic channels and reform in the absence of the pressure communicated by the fluidic channels. The exposed portions assume a convex shaped configuration when subjected to positive pressure and the exposed portions assume a concave shaped configuration when subjected to negative pressure. The topographic members are provided over the exposed portions. The topographic members move based on the deformation of the exposed portions. The strength of adhesion to a counterface varies based on the deformation of the exposed portions.

### BRIEF DESCRIPTION OF DIAGRAMS

This disclosure is illustrated by way of example and not limitation in the accompanying figures. Elements illustrated in the figures are not necessarily drawn to scale, in which like references indicate similar elements and in which:

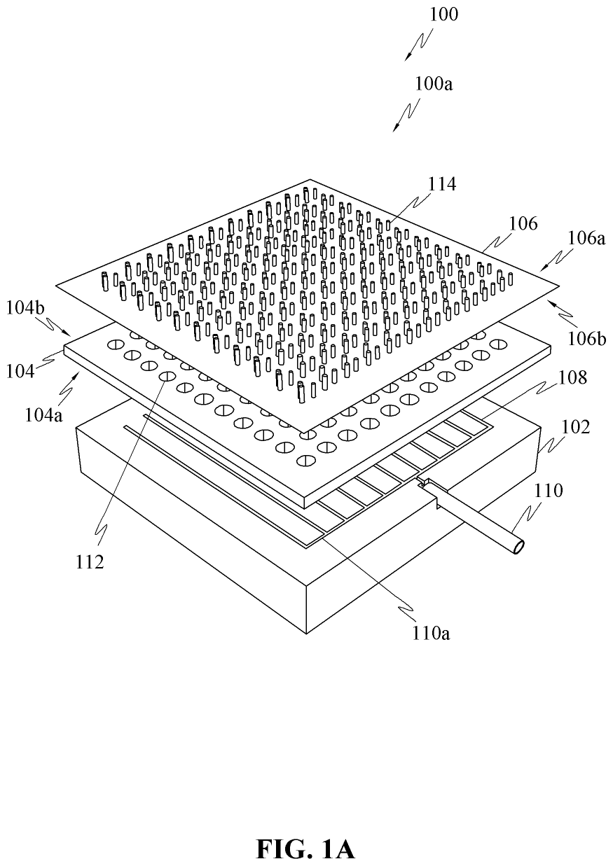

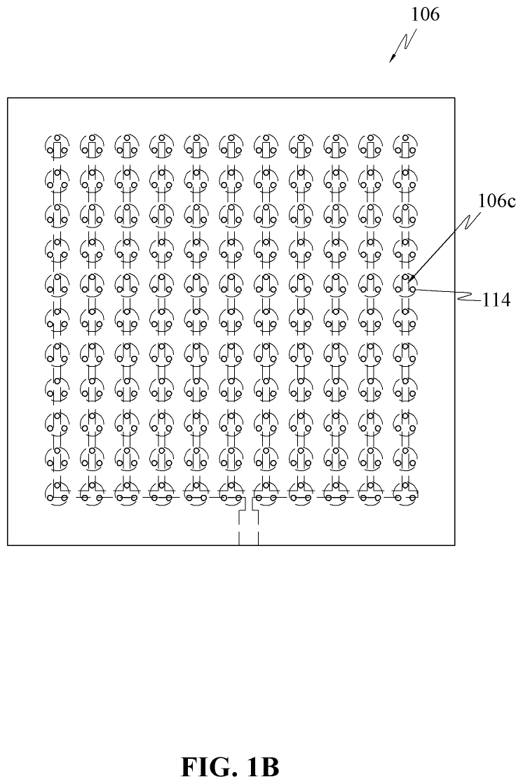

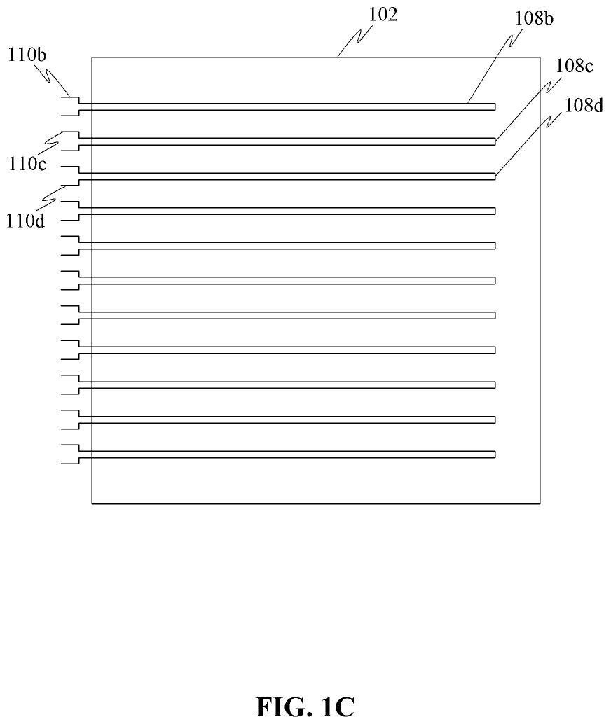

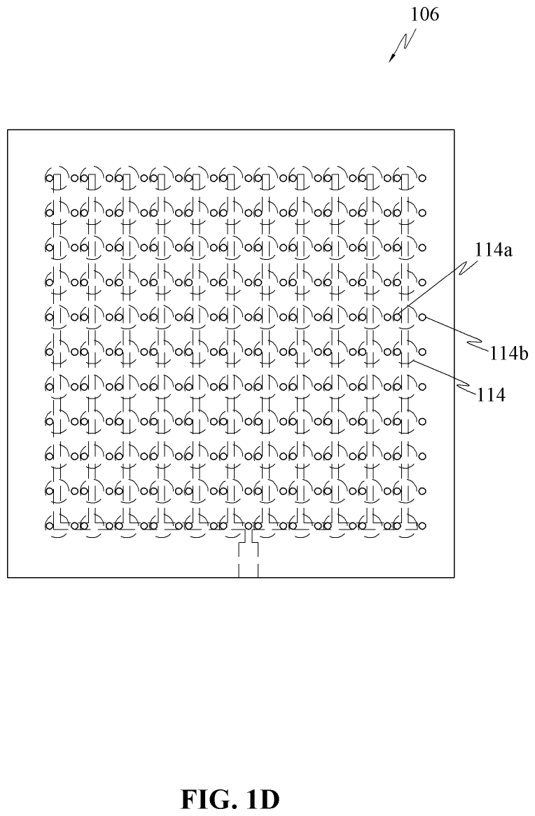

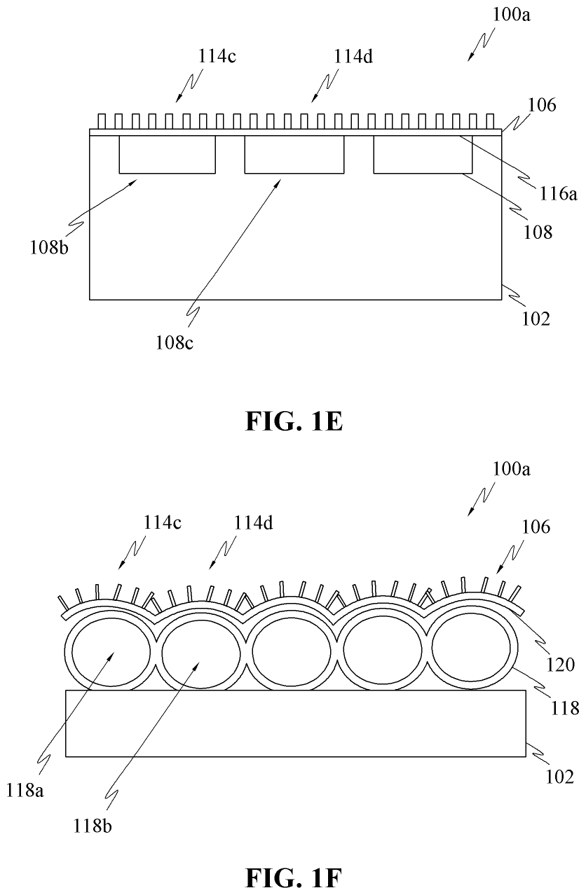

FIG. 1A is a schematic representation of a main body 100a of a system 100, in accordance with an embodiment;

FIG. 1B is a top view of an interface member 106, in accordance with an embodiment;

FIG. 1C is a top view of a channel member 102, in accordance with another embodiment;

FIG. 1D is a top view of the interface member 106, in accordance with another embodiment;

FIG. 1E is a schematic representation of the main body 100a, in accordance with another embodiment;

FIG. 1F is a schematic representation of the main body 100a, in accordance with yet another embodiment;

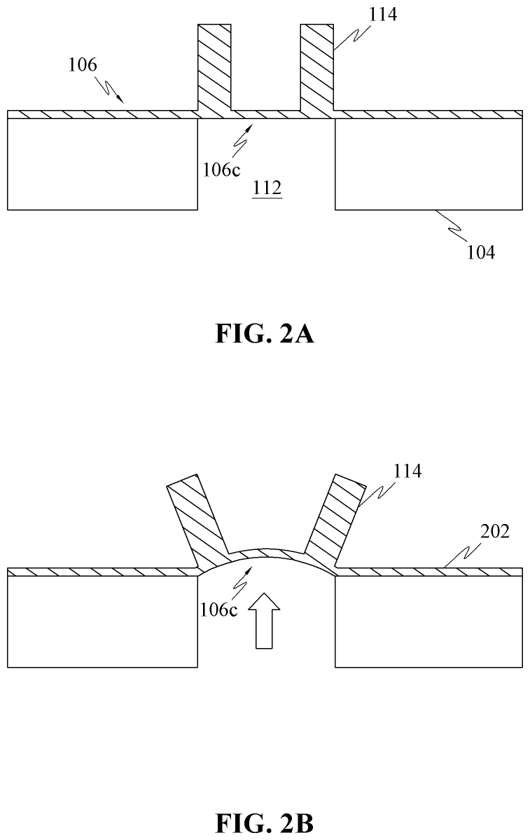

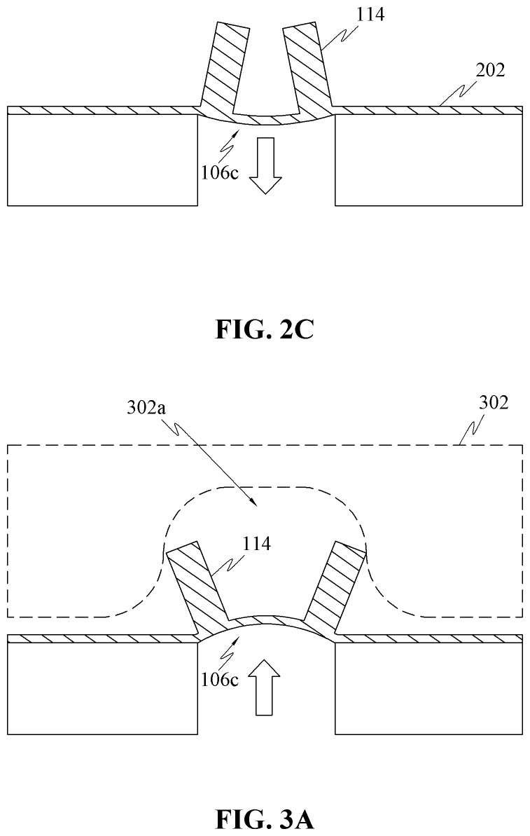

FIG. 2A is a schematic representation of posts 114 in neutral orientation, in accordance with an embodiment;

FIG. 2B is a schematic representation of the posts 114 in convex orientation, in accordance with an embodiment;

FIG. 2C is a schematic representation of the posts 114 in concave orientation, in accordance with an embodiment;

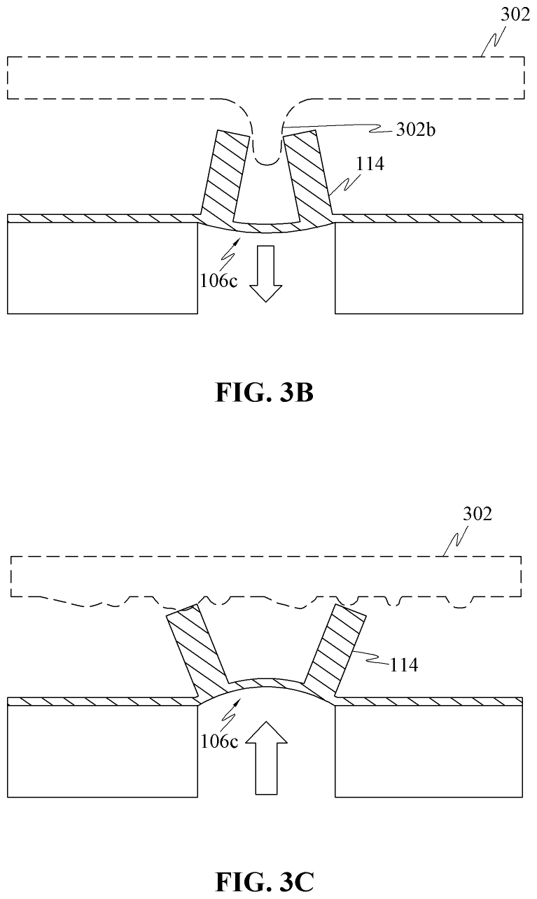

FIG. 3A is a schematic representation of the posts 114 adhering to crevices 302a of an external body 302, in accordance with an embodiment;

FIG. 3B is a schematic representation of the posts 114 adhering to peaks 302b of the external body 302, in accordance with an embodiment;

FIG. 3C is a schematic representation of the posts 114 adhering to a rough surface 302, in accordance with an embodiment;

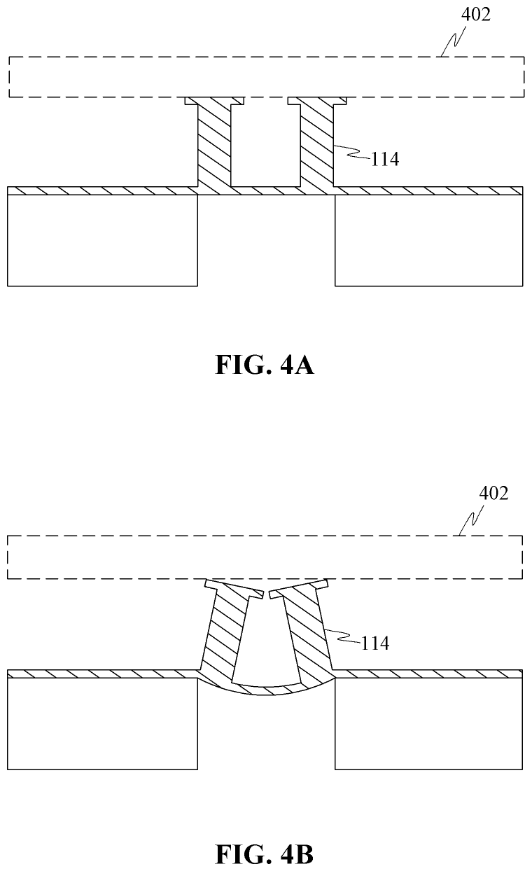

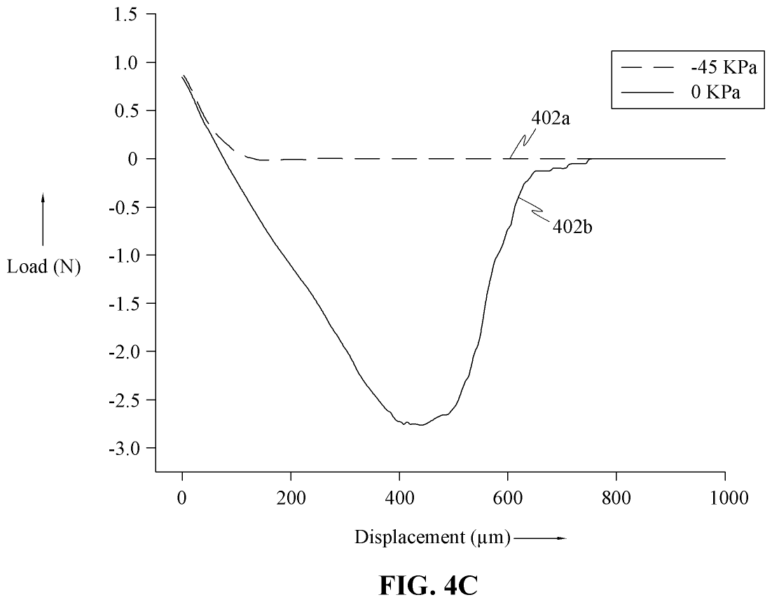

FIG. 4A is the schematic representation of the posts 114 adhering to a smooth surface 402, in accordance with an embodiment;

FIG. 4B is a schematic representation of the posts 114 losing surface contact with the smooth surface 402, in accordance with an embodiment;

FIG. 4C depicts adhesion force to a glass substrate under neutral and vacuum channel pressures, in accordance with an embodiment;

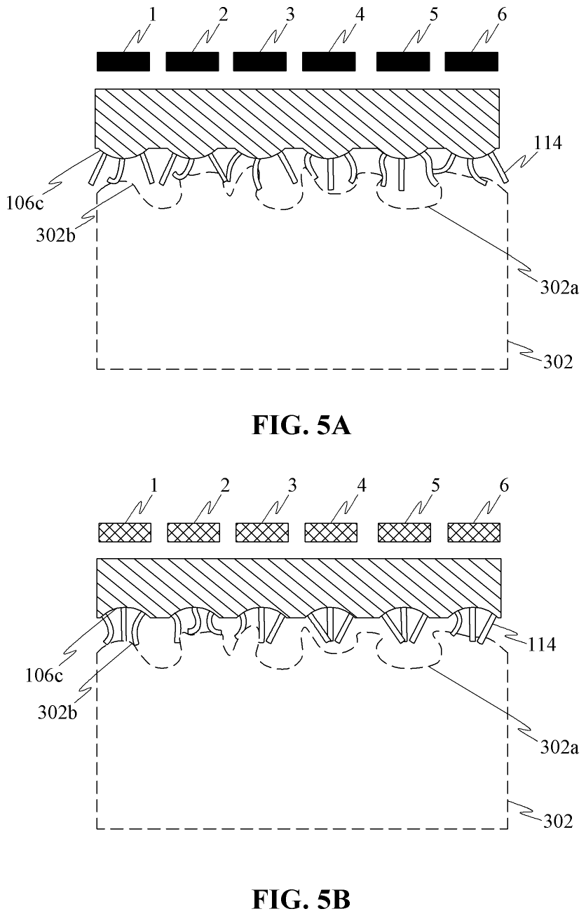

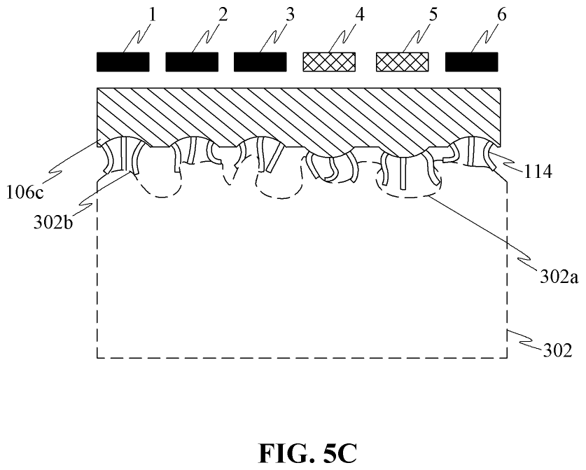

FIG. 5A is schematic representation of orientation of the posts 114 when the system 100 is subjected to only positive external pressure, in accordance with an embodiment;

FIG. 5B is schematic representation of the orientation of the posts 114 when the system 100 is subjected to only negative external pressure, in accordance with an embodiment;

FIG. 5C is schematic representation of the orientation of the post 114 when the system 100 is subjected to both negative and positive external pressures, in accordance with an embodiment;

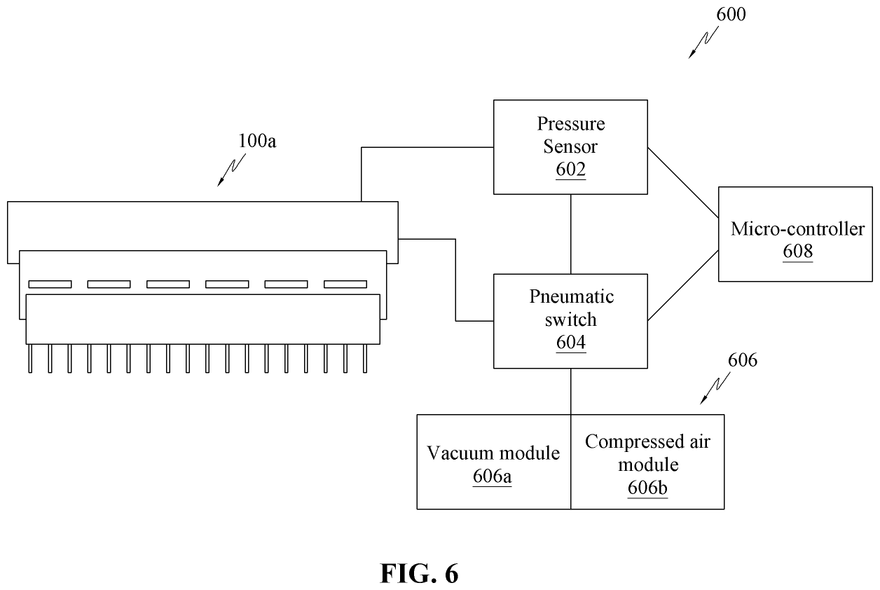

FIG. 6 is a schematic representation of a control system 600, in accordance with an embodiment;

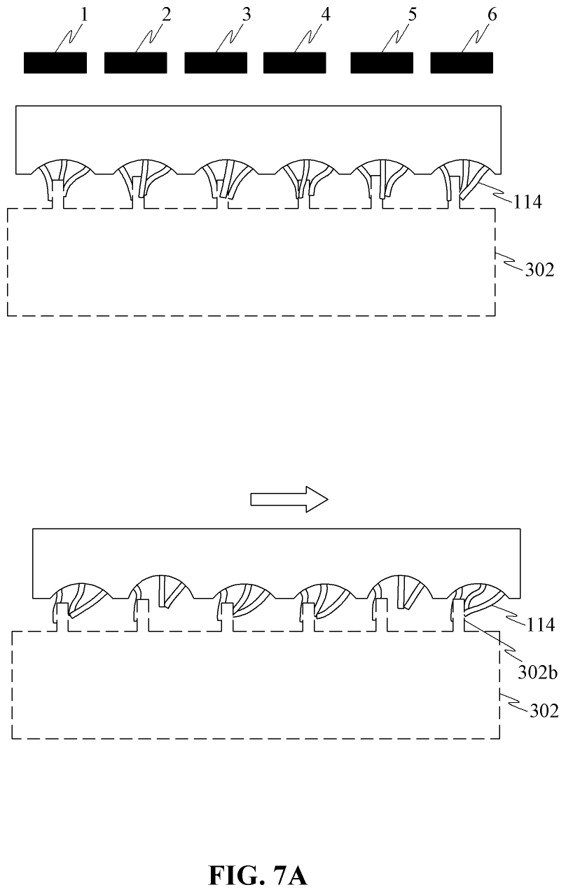

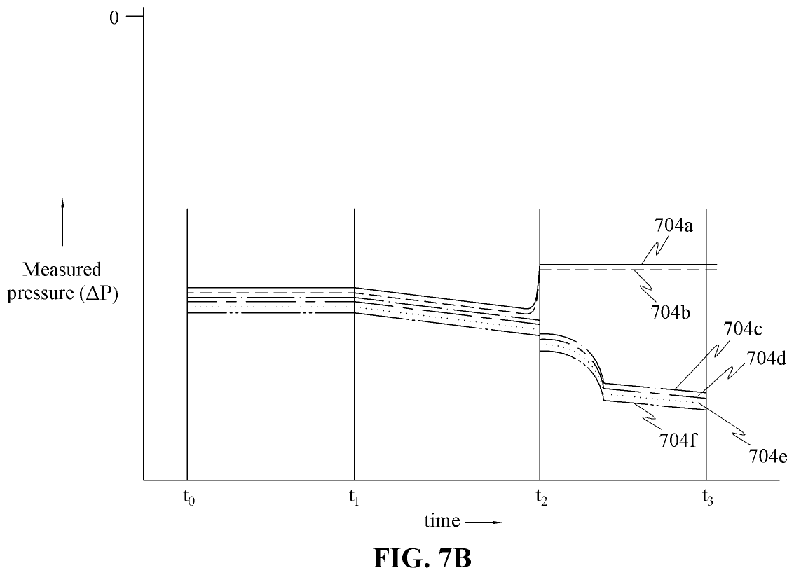

FIG. 7A is a schematic representation depicting the slipping of the posts 114 at hole sites 2 and 5, in accordance with an embodiment;

FIG. 7B is a graphical representation depicting variation of pressure due to slipping of the posts 114, in accordance with an embodiment;

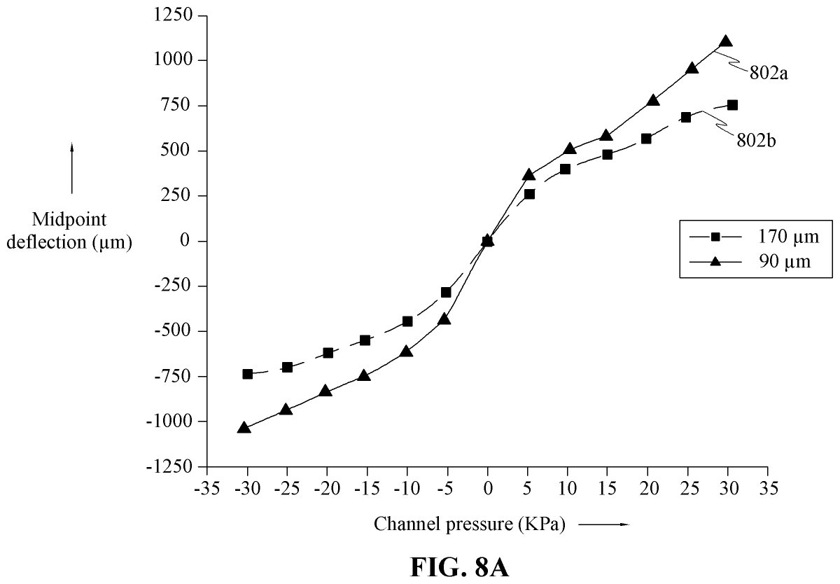

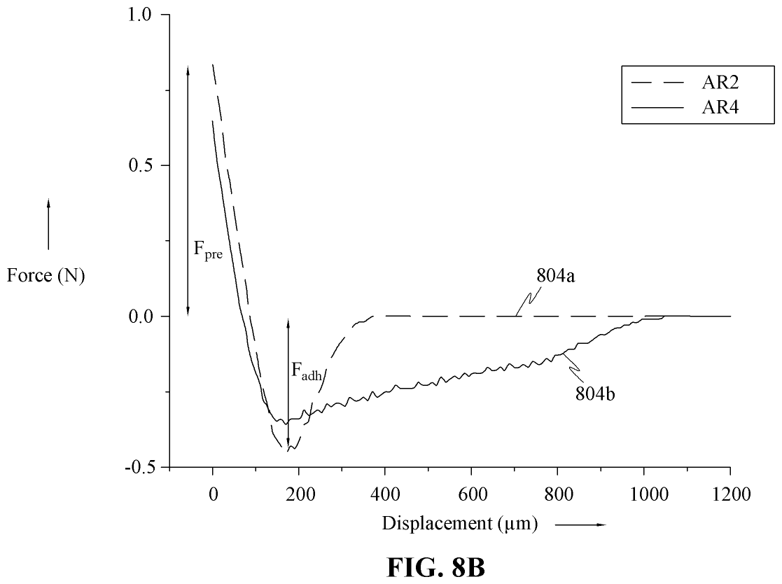

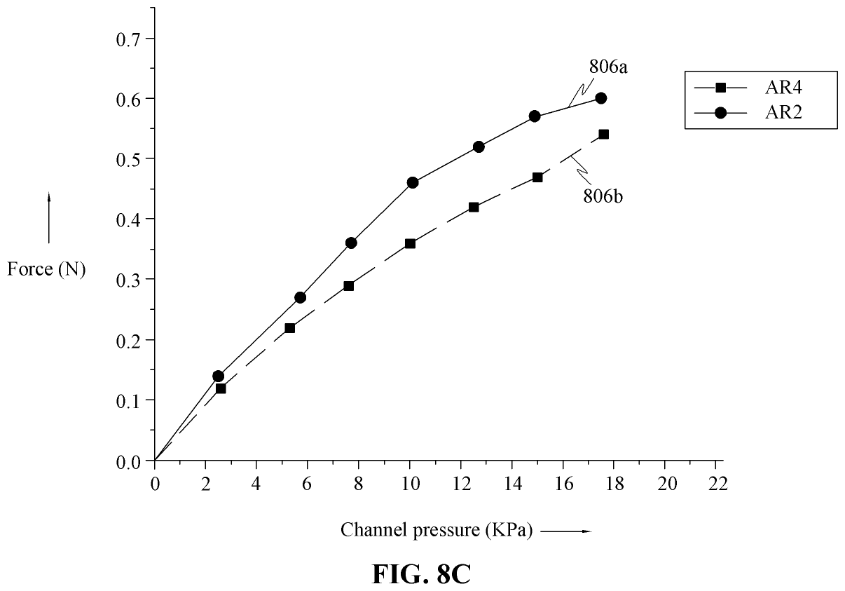

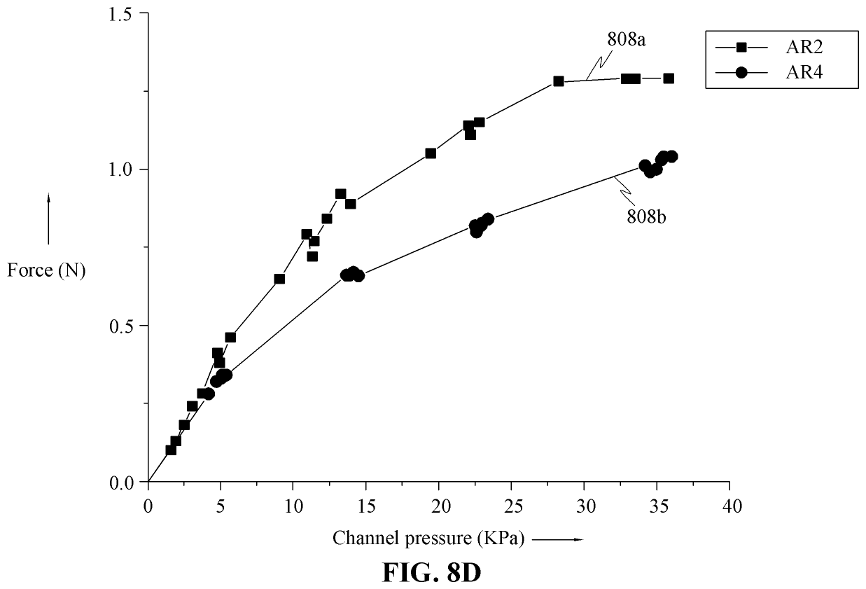

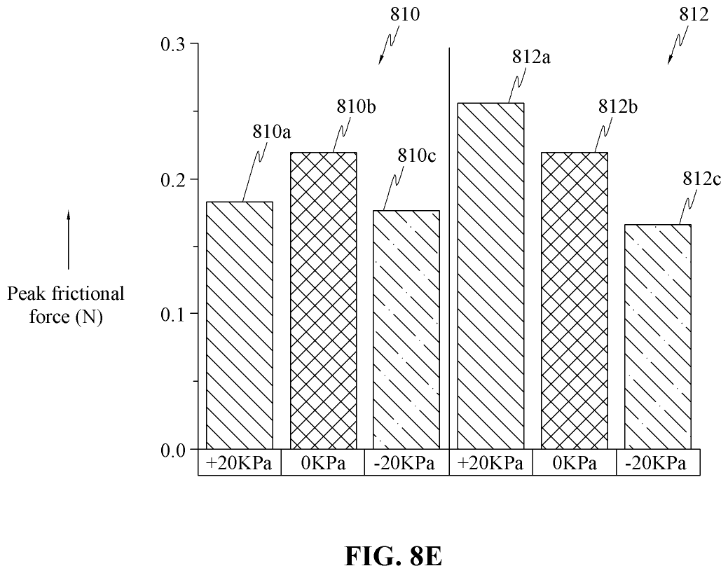

FIG. 8A is a graphical representation depicting deflection of the posts 114 from the neutral state with variation in channel pressure, in accordance with an embodiment;

FIG. 8B is a graphical representation depicting expansion anchor adhesion curves, in accordance with an embodiment;

FIG. 8C is a graphical representation depicting adhesion force (F_{adh}) as a function of channel pressure (kPa), in accordance with an embodiment;

FIG. 8D is a graphical representation depicting retraction gripping adhesion curve, in accordance with an embodiment;

FIG. 8E is a graphical representation depicting frictional force of an active surface system 100 while being pulled across a smooth and a rough surface, in accordance with an embodiment;

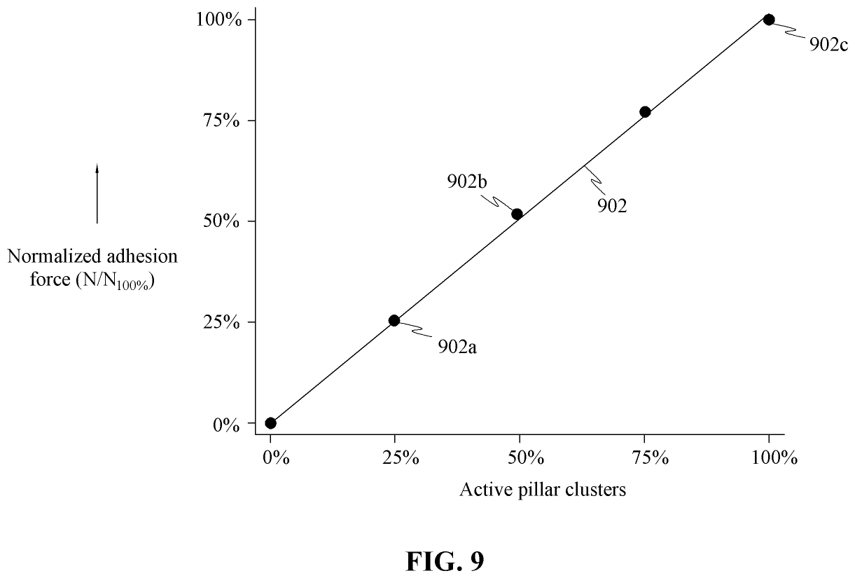

FIG. 9 is a graphical representation depicting linear increase in the adhesion force, with increase in number of quadrants activated, in accordance with an embodiment;

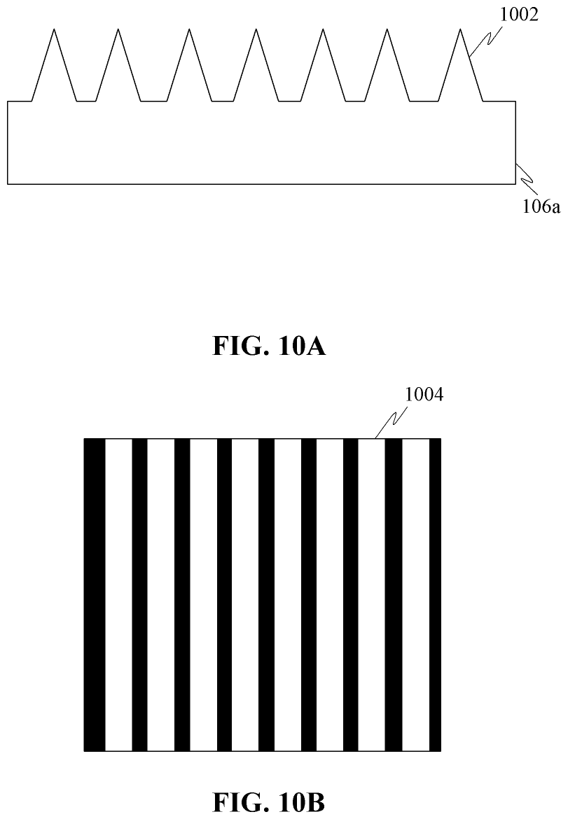

FIG. 10A is a schematic representation of a plurality of cone or pyramid shaped posts 1002, in accordance with another embodiment;

FIG. 10B is a schematic representation of a plurality of straight walls 1004, in accordance with yet another embodiment;

FIG. 10C is a schematic representation of a plurality of straight walls 1006, in accordance with yet another embodiment;

FIG. 10D is a schematic representation of a plurality of irregular shaped topographic members 1008, in accordance with yet another embodiment;

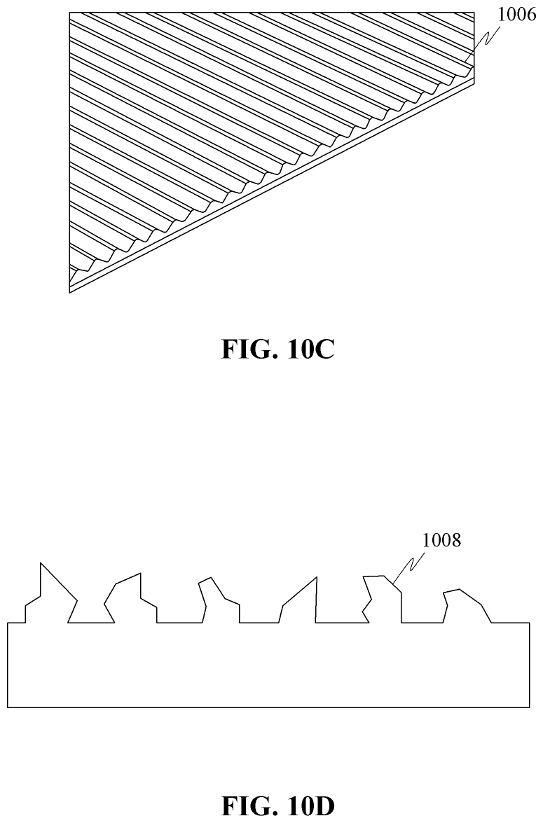

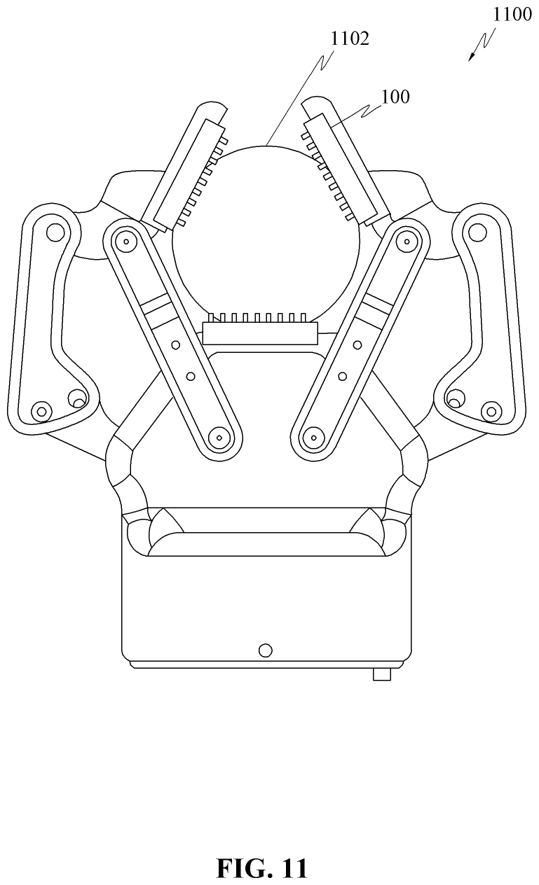

FIG. 11 depicts system 100 adapted on a robotic gripper system 1100 as finger pads, in accordance with an embodiment;

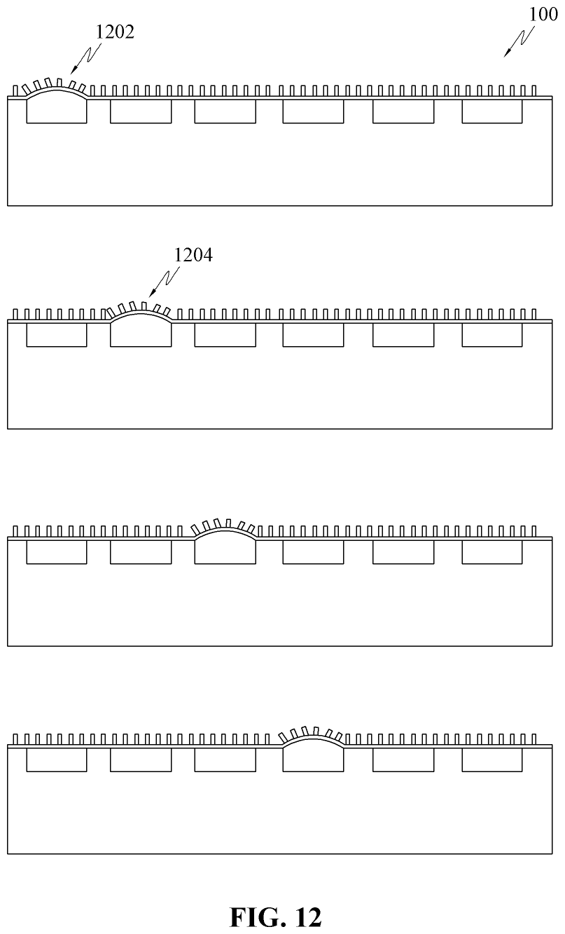

FIG. 12 depicts a locomotion mechanism of the system 100, in accordance with an embodiment; and

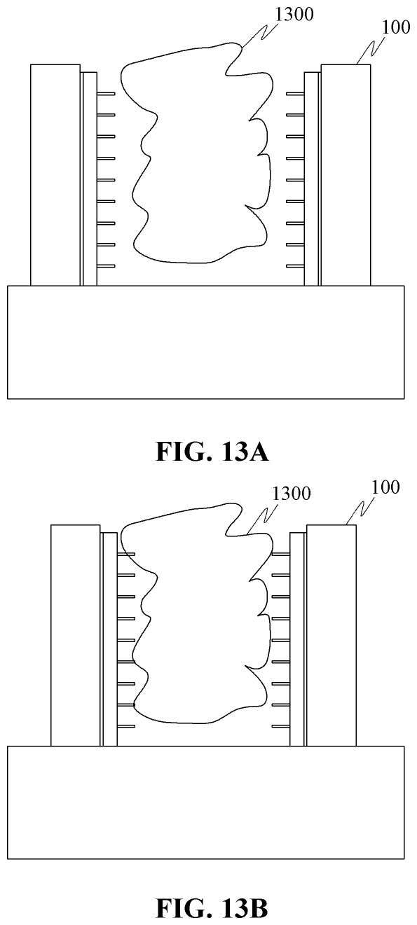

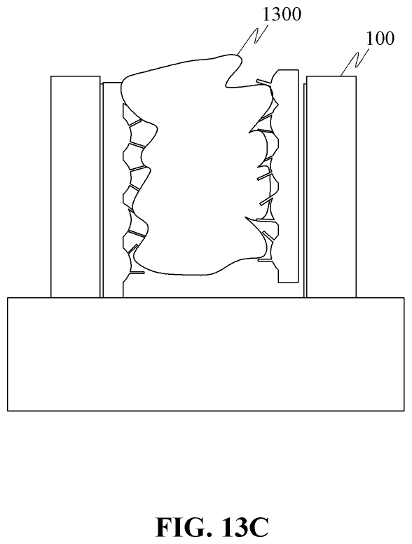

FIGS. 13A-13C are schematic representation illustrating gripping action of the system 100, in accordance with an embodiment.

### DETAILED DESCRIPTION

The following detailed description includes references to the accompanying drawings, which form part of the detailed description. The drawings show illustrations in accordance with example embodiments. These example embodiments are described in enough detail to enable those skilled in the art to practice the present subject matter. However, it may be apparent to one with ordinary skill in the art that the present invention may be practised without these specific details. In other instances, well-known methods, procedures and components have not been described in detail so as not to unnecessarily obscure aspects of the embodiments. The embodiments can be combined, other embodiments can be utilized, or structural and logical changes can be made without departing from the scope of the invention. The following detailed description is, therefore, not to be taken in a limiting sense.

In this document, the terms “a” or “an” are used, as is common in patent documents, to include one or more than one. In this document, the term “or” is used to refer to a non-exclusive “or”, such that “A or B” includes “A but not B”, “B but not A”, and “A and B”, unless otherwise indicated.

It should be understood that the capabilities of the invention described in the present disclosure and elements shown in the figures may be implemented in various forms of hardware, firmware, software, recordable medium or combinations thereof.

Referring to FIG. 1A, a system 100 is provided for controllably establishing adhesion or friction with an external body. The system 100 may achieve reversible and variable interfacial strength of adhesion using a pneumatic or hydraulic mechanism. The system 100 may comprise a main body 100a and a control system 600 (FIG. 6). The main body 100a may comprise a bottom channel member 102, a middle pressure communication member 104 and a top interface member 106.

In an embodiment, the channel member 102 may be configured to comprise a plurality of microfluidic channels 108, wherein, the plurality of microfluidic channels 108 may be connected together by a main channel 110a. The channel member 102 may be in communication with a fluid inlet 110, wherein fluids (hydraulic or pneumatic) may be injected into and removed from the main channel 110a through the fluid inlet 110. The fluid injected into the main channel 110a may be, but not limited to, gas. The channel member 102 may be made using, but not limited to, polymethylmethacrylate (PMMA) sheet. Fluid may be injected into (or removed from) the microfluidic channel 108 to subject the system 100 to positive or negative relative pressure.

In another embodiment, referring to FIG. 1C, the channel member 102 may be configured to comprise a plurality of microfluidic channels 108, wherein, the plurality of microfluidic channels 108 may be independent of each other. That is to say, the plurality of the microfluidic channels may be not connected to each other. Each of the microfluidic channels 108 may be in communication with a plurality of fluid inlets 110. As an example, channel 108b may be in communication with fluid inlet 110b, channel 108c may be in communication with fluid inlet 110c, channel 108d may be in communication with fluid inlet 110d and so on. Each of the microfluidic channels 108 may be subjected to negative or positive external pressure independently. As an example, the microfluidic channel 108b may be subjected to positive pressure, the microfluidic channel 108b may be subjected to negative pressure and so on.

In an embodiment, referring to FIG. 1A, the pressure communication member 104 may be configured to comprise a first side 104a and a second side 104b opposite the first side 104a. The second side 104b interfaces with the interface member 106 and the first side 104a may be engaged with the channel member 102. The pressure communication member 104 may comprise a plurality of through holes 112 extending between the first side 104a and the second side 104b. The holes 112 may be circular having a radius of 1.5 mm. The holes may be of shapes other than circular, such as, but not limited to any polygonal shape (square, rectangular).

The holes 112 may function to localize adhesion sites, improve rigidity of the surface and create a perpendicular edge for posts 114 to move (rotate) around the holes 112. During construction, the pressure communication member 104 may be placed above the channel member 102 in such a way that the plurality of holes 112 may be positioned above the plurality of microfluidic channels 108.

In the embodiment, referring to FIG. 1A and FIG. 1B, the interface member 106 may comprise a bottom portion 106b and a top portion 106a, wherein the bottom portion 106b may be configured to comprise exposed portions 106c. The exposed portions 106c may be the area on the bottom portion 106b of the interface member 106 that may be exposed to the holes 112.

The top portion 106a may be configured to comprise a plurality of raised topographic members. In an embodiment, referring to FIG. 3A, the topographic members may be straight posts 114.

In another embodiment, referring to FIG. 4A, the topographic members may be mushroom shaped posts.

In yet another embodiment, referring to FIG. 10A, the topographic members may be cone or pyramid shaped posts 1002. The base of the cone shaped posts 1002 may be circular.

In yet another embodiment, referring to FIG. 10B, the topographic members may be parallel walls 1004.

In yet another embodiment, referring to FIG. 10C, the topographic members may be walls 1006. The walls 1006 may extend the top surface of the interface member.

In yet another embodiment, referring to FIG. 10D, the topographic members may be irregular shaped topographic members 1008. These irregular shaped features can be hard inorganic/ceramic particles, such as silicate particles or sandpaper

Embodiments are hereinafter explained largely in the context of straight posts 114. However, the concepts are adaptable to other configurations of the topographic members.

Referring to FIG. 1B, each of the exposed portions 106c may be configured to comprise one cluster of posts 114 with three posts 114 per cluster. That is to say, the top portion 106a corresponding to each of the exposed portion 106c may have three posts 114 disposed in a triangular configuration. The posts 114 may be movable and may move along with the exposed portions 106c to assume a concave or a convex orientation when subjected to external pressure, which is described in detail below. During construction, the interface member 106 may be placed above the pressure communication member 104 in such a way that the posts 114 may be positioned within the boundary defined by each of the holes 112, but away from centre of each of the holes 112. The radius of the posts 114 may be 0.40 mm and the height may be 0.80 mm or 1.60 mm. The posts 114 may also have other dimensions.

In another embodiment, the number of posts 114 per cluster may be two (02). That is to say, each of the exposed portion 106c may have two posts 114. The posts 114 may be movable and may move along with the exposed portions 106c to assume the concave or the convex orientation when subjected to external pressure.

In yet another embodiment, referring to FIG. 1D, apart from movable posts 114a, there may be non-movable posts 114b provided over the non-exposed portions of the interface member 106. The movement of the movable posts 114a relative to the non-movable posts 114b may provide the requisite adhesion to a counterface.

Having discussed the constructional configuration of the main body 100a of the system 100, the various interlocking mechanisms of the posts 114 are discussed hereunder.

In an embodiment, the posts 114 may assume various orientations with respect to the interface member 106 to participate in adhesion to the external body (counterface). The exposed portions 106c, and thereby, the posts 114 may assume a neutral orientation, concave orientation or convex orientation.

In yet another embodiment, referring to FIG. 1E, the main body 100a may comprise the bottom channel member 102 and the top interface member 106. That is to say, the main body 100a may not comprise the middle pressure communication member 104. In such a configuration, when the microfluidic channels 108 are subjected to the negative pressure, a first exposed area 116a may retract causing the plurality of posts 114 to assume the concave shape. That is to say, when the negative pressure is applied to the system 100, the first exposed portion 116a positioned above the microfluidic channels 108 may retract causing the posts 114 within one microfluidic channel 108 to tilt towards each other. As an example, posts 114c above the microfluid channel 108b may tilt towards each other, posts 114d above the microfluid channel 108c may tilt towards each other and so on. The first exposed area 116a may be the area exposed to the microfluidic channels 108.

In the embodiment, when the microfluidic layers 108 are subjected to positive pressure, the first exposed area 116a may expand causing the plurality of posts 114 to assume the convex shape. That is to say, when the positive pressure is applied to the system 100, the first exposed portion 116a positioned above the microfluidic channels 108 may expand causing the posts 114 within one microfluidic channel 108 to tilt away from each other. As an example, posts 114c above the microfluid channel 108b may tilt away from each other, posts 114d above the microfluid channel 108c may tilt away from each other and so on.

In yet another embodiment, referring to FIG. 1F, the main body 100a may comprise the bottom member 102, a plurality of circular tubes 118 arranged parallel to each other and the flexible top interface member 106. In such a configuration, the positive or the negative pressure may be applied to the plurality of the tubes 118. When the tubes 118 are subjected to the negative pressure, the tubes 118 may contract, causing the posts 114 to assume the concave shape. That is to say, when the negative pressure is applied to the main body 100a, then a tube exposed area 120 may retract, causing the posts 114 within one tube 118a to tilt towards each other. As an example, posts 114c above the tube 118a may tilt towards each other, posts 114d above the tube 118b may tilt towards each other and so on. The tube exposed area 120 may be the exposed area above each of the plurality of the tubes 118.

When the tubes 118 are subjected to positive pressure, the tubes 118 may expand, causing the posts 114 to assume the convex shape. That is to say, when the positive pressure is applied to the main body 100a, then the tube exposed area 120 may expand, causing the posts 114 within one tube 118a to tilt away from each other. As an example, posts 114c above the tube 118a may tilt away from each other, posts 114d above the tube 118b may tilt away from each other and so on.

Referring to FIG. 2A, when no external pressure is applied to the main body 100a of the system 100, the posts 114 may assume a neutral state. That is to say, the posts 114 may be oriented perpendicular to the exposed portion 106c.

Referring to FIG. 2B, when a positive external pressure is applied to the system 100, the exposed portion 106c, and thereby the posts 114, may assume a convex orientation. Such a configuration may be possible due to the flexibility of the interface member 106. When the positive external pressure is applied to the main body 100a of the system 100, the exposed portion 106c positioned above the hole 114 may expand causing the posts 114 within a cluster to tilt away from each other. The titled posts 114 may be at an acute angle with unexposed portion 202. The unexposed portion 202 may the stationary neighbouring area around the exposed portion 106c.

Referring to FIG. 2C, when negative external pressure is applied to the main body 100a of the system 100, the exposed portion 106c, and thereby the posts 114 may assume a concave orientation. When the negative external pressure is applied to the system 100, the exposed portion 106c positioned above the hole 114 may retract causing the posts 114 within a cluster to tilt towards each other. The titled posts 114 may be at an obtuse angle with the unexposed portion 202. In an embodiment, the negative pressure may tilt the posts 114 to such an extent that the posts 114 generally align with the plane of the unexposed portion 202 of the interface member 106 or may even retract underneath the plane of the unexposed portion 202 of the interface member 106.

The orientation of the posts 114 (neutral, convex, concave) may be configured based on the type of external body (counterface) 302 onto which the system 100 needs to establish adhesion. Referring to FIG. 3A-3C and FIG. 4A, the external body 302 may have a smooth surface (even surface) 402 or a rough surface (uneven surface) 302. The rough surface 302 may have crevices 302a and/or peaks 302b.

In an embodiment, referring to FIG. 3A, when the external body 302 has crevices 302a, the posts 114 may assume the convex orientation to grip onto the external body 302. In such a scenario, the system 100 may be subjected to positive external pressure and the exposed portion 106c may expand causing the posts 114 to tilt outwards (convex orientation) into the walls of the crevices 302a. The posts 114 may be pressed against the walls of the crevices 302a establishing adhesion to the counterface surface 302. When the positive external pressure is removed, the posts 114 may tilt back into the neutral orientation allowing a reduction in adhesion strength. That is to say, when the external pressure is removed, the system 100 may experience reduced adhesion strength with the external body 302.

In an embodiment, referring to FIG. 3B, when the external body 302 has peaks 302b, the posts 114 may assume the concave orientation to grip onto the external body 302. In such a scenario, the system may be subjected to negative external pressure and the exposed portion 106c may retract causing the posts 114 to tilt inward (concave orientation). The posts 114 may grip the protruding roughness (peak 302b) establishing adhesion to the surface 302. When the negative external pressure is removed, the posts 114 may tilt back into the neutral state causing reduced adhesion strength (reverse adhesion). That is to say, when the external pressure is removed, the system 100 may lose the adhesion with the external body 302.

In an embodiment, referring to FIG. 4A, when the external body has a smooth surface 402, the posts 114 may assume the neutral orientation to grip onto the external body 402. For the posts 114 to grip onto the smooth counterface surface 402, the posts 114 may be configured to be mushroom shaped. The mushroom shaped posts 114 may comprise a vertical part and a lateral part (mushroom head), wherein the lateral part may extend laterally from the vertical part. The lateral part of the mushroom shaped posts 114 may be configured to interface with the external body 402. No external pressure may be provided when the system 100 has to grip onto the smooth surface 402, such as a glass substrate. The mushroom head of the posts 114 may adhere to surfaces using van der Waals force (dry adhesion). Referring to FIG. 4B, for reverse adhesion, the system 100 may be subjected to external pressure. As an example, when the system 100 is subjected to the negative external pressure, the posts 114 may tilt inwards. The contact area necessary for the posts 114 to stick to the smooth surface of the external body 402 may be diminished, thereby switching the device surface from a ‘sticky’ (FIG. 4A) to ‘non-sticky’ state (FIG. 4B). In conclusion, when the system 100 is subjected to external pressure, the system 100 may lose adhesion with the external body 402 and when no external pressure is supplied to the system 100, the system 100 may establish adhesion with the external body 402.

FIG. 4C shows adhesion force to a glass substrate under neutral and vacuum channel pressures. Without any applied pressure, the dry-adhesive topography provides strong adhesion (2.9N±0.1N) while, under vacuum, there is almost no adhesion force (0.02N±0.0N). Because the negative pressure rotates the posts inward, the contact area necessary for the dry-adhesives to stick is diminished, thereby switching the surface from a ‘sticky’ to ‘non-sticky’ state.

Moving on, in an embodiment for mechanical interlock, consider subjecting the system 100 to only positive external pressure. In such a scenario, the exposed portion 106c may expand causing the cluster of posts 114 to assume the convex orientation. Referring 6 to FIG. 5A, the cluster of posts 114 positioned at the edge of the hole sites 1, 2, 3, 4, 5 and 6 may assume convex orientation and may try to press against the walls of the crevices 302a. Only the posts 114 at the hole sites 3, 4, and 5 may be successful in getting adhered to the crevices 302a. The posts 114 at the hole sites 1, 2 and 6 may not be successful in becoming adhered within the crevices 302a due to the presence of the peaks 302b. That is to say, posts 114 at the hole sites 1, 2 and 6 may exhibit weak adhesion and the posts at the hole sites 3, 4 and 5 may exhibit strong adhesion.

Now consider subjecting the system 100 to only negative external pressure. In such a scenario, the exposed portion 106c may retract causing the cluster of posts 114 to assume the concave orientation. Referring to FIG. 5B, the cluster of posts 114 positioned at the edge of the hole sites 1, 2, 3, 4, 5 and 6 may assume concave orientation and may try to grasp the peaks 302b. Only the posts 114 at the hole sites 1, 2 and 3 may be successful in grasping the peaks 302b. The posts 114 at the hole sites 4, 5 and 6 may not be successful in grasping any peaks due to the presence of the crevices 302a. That is to say, posts 114 at the hole sites 1, 2 and 3 may exhibit a strong adhesion and the posts at the hole sites 4, 5 and 6 may exhibit weak adhesion.

Both the above explained scenarios may not result in an optimized device adhesion to a counterface. Referring to FIG. 5C, a combination of retraction and expansion of the exposed portion 106c may be utilized for establishing an optimized adhesion of the system 100 with the external body 302. In such a scenario, some of the microfluidic channels 108 may be subjected to negative external pressure and some of the microfluidic channels 108 may be subjected to positive external pressure. When the hole sites 1, 2, 3 and 6 are subjected to negative external pressure, the posts 114 at these hole sites may assume the concave orientation and may grasp the peaks 302b. At the same time, when the hole sites 4 and 5 are subjected to positive external pressure, the posts 114 at these hole sites may assume the convex orientation and may get adhered to the crevices 302a. Consequently, posts 114 at the hole site 1, 2, 3, 4, 5 and 6 may exhibit a strong adhesion resulting in optimized adhesion. That is to say, the combination of expansion of the exposed portion 106c at hole sites 4 and 5 and retraction of the exposed portion 106c at hole sites 1, 2, 3 and 6 may result in an higher overall adhesion strength.

Having discussed the various interlocking mechanisms of the posts 114, the architectural configuration and the working of the control system 600 are discussed hereunder.

In an embodiment, referring to FIG. 6, the main body 100a of the system 100 may be configured to be in communication with the control system 600. The control system 600 may function to provide the microfluidic channels 108 with negative external pressure or positive external pressure, sense the variation of pressure at the plurality of the microfluidic channels 108 and so on. The control system 600 may be configured to comprise a plurality of pressure sensors 602, pneumatic switches 604, a controller module (not shown) and a pneumatic pressure system 606. The controller module may further comprise of a microcontroller 608. The plurality of pressure sensors 602, the pneumatic switch 604 and the pneumatic pressure system 606 may be controlled by the microcontroller 608.

In an embodiment, the plurality of pressure sensors 602 may be configured for pressure measurements in the microfluidic channels 108 and may transmit the signal to the microcontroller 608. The microcontroller 608 may determine the pressure variation in the microfluidic channels 108 and may provide instructions to the pneumatic pressure system 606 to control the pressure in the microfluidic channels 108.

The plurality of pressure sensors 602 with the microcontroller 608 may monitor the mechanical state of individual microfluidic channels 108. That is to say, the pressure sensors 602 may determine whether the main body 100a has successfully established mechanical adhesion or whether there was a local failure of adhesion. Local failure of adhesion may be due to the slipping of the posts 114. The slipping of the posts may cause variation in the pressure due to the further retraction or expansion of the exposed portion 106a. As an example, referring to FIG. 7A, at a time t₁, when the posts 114 are subjected to negative external pressure, the posts 114 may assume the concave orientation to grasp the peaks of the external surface 302. At time t₂, due to local failure, the posts 114 at hole site 2 and 5 may slip from the peaks 302b causing more retraction of the exposed portion 106a at the respective sites. Consequently, the pressure sensors 602 may detect a pressure increase (vacuum loss) in the respective microfluid channel 108 and may transmit the signal to the microcontroller 608. The microcontroller 608 may instruct the pneumatic pressure system 606 to increase the vacuum pressure in the remaining microfluidic channels 108 respective to the posts 114 at the hole sites 1, 3, 4 and 6. The vacuum pressure at these sites may be increased, so that the posts 114 at the hole sites 1, 3, 4 and 6 may establish strong adhesion to the external body 302. The sensory feedback enables the system 100 to be ‘smart’ and actively respond to adhesion conditions by constantly adjusting pressure to optimize device adhesion. Machine learning algorithms may be utilized for interpreting real-time pressure sensing data to predict adhesion failure. The active surfaces can also sense pressure change, and thereby respond to an external trigger of touch contact. This sensory feedback enables the surfaces to be ‘smart’ and actively respond to adhesion conditions by constantly adjusting differential pressure to optimize overall adhesion.

FIG. 7B is a graphical representation depicting variation of pressure due to slipping at hole sites 2 and 5. Lines 704a and 704b depicts the increase in pressure due to slipping of posts 114 at hole sites 2 and 5. Lines 704c, 704d, 704e and 704f depict the increase in the vacuum pressure at the hole sites 1, 3, 4 and 6 to compensate the slipping at the hole sites 2 and 5.

The pneumatic pressure system 606 may further comprise a compressed air module 606b and vacuum module 606a for providing positive external pressure and negative external pressure, respectively. The vacuum module 606a may be configured to comprise an air pump (not shown) for providing the negative external pressure to the microfluidic channels 108 by suction and the compressed air module 606b may be configured to comprise compressed air for providing the positive external pressure to the microfluidic channels 108 by injecting air into the microfluidic channels 108.

In an embodiment, the microcontroller 608 may control the pneumatic switch 604 to provide compressed air (positive pressure) into the main body 100a or a vacuum (negative pressure) from the main body 100a using the air pump.

Having discussed the architectural configuration and the working of the control system 600, various experimental results are discussed hereunder.

FIG. 8A is a graphical representation depicting the midpoint deflection of the interface member 106 from the neutral state (0 μm) with variation in channel pressure. The experiments to study the displacement of the interface 106 with the applied channel pressure (positive and negative) were conducted on interface member 106 with two different thickness: one interface member 106 with 170 μm thickness and other interface member 106 with 90 μm thickness. The x-axis of the graph represents the channel pressure (kPa) and the y-axis represents the midpoint deflection of the posts (μm). A curve 802a depicts the displacement of a 90 μm thick interface member 106 and a curve 802b depicts the displacement of a 170 μm thick interface member 106. The slope inflection points at |5 kPa| are expected to be caused by mechanical yielding of the interface member 106. The experimental results show that the interface member 106 with lesser thickness (90 μm) showed more midpoint defection as compared to the interface member 106 with greater thickness (170 μm).

FIG. 8B is a graphical representation depicting expansion anchor adhesion curves. The expansion anchor adhesion curves depict the force experienced by the posts 114 with the variation in the positive deflection of the posts 114 from the neutral state (0 μm), when subjected to the positive external pressure. The experiments to study the pull-off adhesion curves for expansion anchoring were conducted for posts 114 of two different height: 0.80 mm (P1) and 1.60 mm (P2) posts. The x-axis of the graph represents the displacement (μm) of the posts 114 and the y-axis represents the force (N). A curve 804a depicts the expansion anchor adhesion curve for P1 and 804b depicts the expansion anchor adhesion curve for P4. The expansion anchor adhesion curves for posts P1 and P2 depict an initial force (Fₚᵣₑ) and an adhesion force (F_{adh}), wherein the initial force may be due to the interlocking of the posts 114 to the walls of the crevices 302a and adhesion force may be the attractive force between the posts 114 and the walls of the crevices 302a. The adhesion force (F_{adh}) may be maximized before slowly reducing, wherein the slow reduction of the adhesion force (F_{adh}) is due to the loss of contact of the posts 114 with the walls of the crevices 302a. From the graph it may be understood that the P2 posts retain adhesion at higher displacements due to relatively taller posts having a larger contact time with the walls of the crevices 302a.

FIG. 8C is a graphical representation depicting the pull-off adhesion force (F_{adh}) for a device as a function of channel pressure (kPa). The experiments to study the effect of channel pressure on the adhesion force were conducted for P1 and P2 posts. The x-axis of the graph represents the channel pressure (kPa) and the y-axis represents the adhesion force (N). A curve 806a depicts the variation of the adhesion force for P1 posts and 806b depicts the variation of the adhesion force for P2 posts. From the graph it may be understood that, as the channel pressure increases, increasing expansion of the exposed portion 106c may create a larger pressure into the walls of the crevices 302, resulting in a linear increase in maximum adhesion. Further, P1 posts may show a higher adhesion force. This may be because, from the experiments it was seen that the P2 posts bend while the P1 posts remain rigid, transferring the tilt of the posts into higher pressure.

FIG. 8D is a graphical representation of pull-off adhesion curves for a device in retraction gripping. The retraction gripping adhesion curve depict the adhesion force, when subjected to negative external pressure. The experiments to study the effect of channel pressure on the adhesion force were conducted for P1 and P2 posts. The x-axis of the graph represents the negative channel pressure (kPa) and the y-axis represents the adhesion force (N). A curve 808a depicts the variation of the adhesion force for P1 posts and 808b depicts the variation of the adhesion force for P2 posts. From the graph it may be understood that, as the negative channel pressure decreases (increase in vacuum), adhesion force increases. However, compared to convex expansion anchoring (FIG. 8C), the increase in adhesion force diminishes with greater channel pressure. This may be because post rotation is reduced in favour of vertical deflection when a large vacuum is applied. Further, P1 posts may show a higher adhesion force.

FIG. 8E is a graphical representation depicting frictional force of the active surface system 100 while being pulled across a smooth (e.g.: cast acrylic) and a rough surface (e.g.: 120 grit sandpaper), with Y-axis representing the frictional force (N). From the graph it may be seen that, for the smooth surface 810 the frictional force may be highest when the posts 114 are in the neutral state and the frictional force may be lowest when the posts 114 are in the concave or the convex state. Bar 810b depicts the frictional force peak when the posts 114 may be in the neutral state and bars 810a and 810c depict the frictional force peak when the posts 114 may be in the convex state and the concave state respectively. For the rough surface 812, when the channel pressure was increased to +20 KPa, an 18% more resistance was observed, whereas, when the channel pressure was decreased to −20 KPa (bar 812c), a reduction in resistance by 23% (bar 812c) was observed. As a result, the coefficient of friction of the device surface is actively changed due to the underlying channel pressure.

Having discussed the various experimental results, the fabrication of the main body 100a is discussed hereunder.

In an embodiment, referring to FIG. 1A, the interface member 106 may be fabricated from an elastomer material, and by casting of PDMS (Polydimethylsiloxane) silicone such as, but not limited to, DOW CORNING SYLGARD 184 into a CNC machined mould.

In the embodiment, the pressure communication member 104 may be fabricated from a double sided acrylic adhesive closed cell foam and the plurality of holes 112 may be formed by laser cutting. The bottom channel member 102 may be CNC machined from a PMMA sheet and may have a thickness, but not limited to, 3.175 mm.

In the embodiment, each face of the main body 100a may be plasma oxidized for 1 minute to increase the surface energy for promoting adhesion. Further, fluid inlet 110 may be formed from a 20-gauge syringe needle (plastic or metallic) and may be inserted into the bottom channel member 102 with a coating of epoxy to form a seal.

In another embodiment, referring to FIG. 3C, the mushroom shaped post may be fabricated using a secondary dip process. A blank mould with a depth of 100 μm may be filled with PDMS and the membrane posts may be dipped into the PDMS. The membrane may be pressed (posts down) onto a glass substrate, cured, and peeled from the glass substrate.

Having discussed the fabrication of the main body 100a, the method for optimizing overall adhesion is discussed hereunder.

Consider the plurality of independent microfluidic channels 108 (or the walls 114) divided into four quadrants. Each of the quadrant may comprise one or more number of microfluidic channels 108. The optimized overall adhesion strength of the system 100 may be achieved by the activation of all the quadrants. Referring to FIG. 9, a line 902 depicts the linear increase in the pull-off adhesion force, with increase in number of quadrants activated. That is to say, as varying numbers of quadrants are activated, adhesion force increases linearly. The number of active array clusters is proportional to the maximum adhesion. At a first point 902a, only one quadrant may be activated. From the graph it can be understood that the overall adhesive strength is minimum at the first point 902a. At a second point 902b, where the number of quadrants activated may be 2, the overall adhesive strength can be seen increasing. Further, at the point 902c, where all the quadrants may be activated, the overall adhesive strength can be seen to be maximum.

Moving on the application, system 100 may be used in biomedical applications. The system 100 may be used for reversible adhesion to biological tissue, such as skin or internal organs, for medical skin devices such as bioelectrodes, drug delivery patches, surgical robotics and so on.

Referring to FIG. 11, the system 100 may be adapted on a robotic gripper system 1100 as finger pads. The posts 114 on the system may adhere to an external body 1102 for holding, tightening or handling the external body 1102. The relative strength of the adhesion of the interface members 106 on plurality of the robotic arms may be manipulated. That is to say, if the adhesive strength of the interface member 106 on one of the robotic arms reduces, then the adhesive strength of the interface member 106 of the remaining robotic arms may be increased to optimize adhesion to the external body 1102.

The system 100 may further be used for locomotion, wherein the system 100 may move from one point to another relative to the external surface. The locomotion of the system 100 may be achieved through periodic, peristaltic actuation of the posts 114. The actuation of the posts 114 may control locomotion relative to the external surface. Referring to FIG. 12, the system 100 may establish adhesion to the external surface by subjecting the posts 1202 to positive pressure. Then the positive pressure subjected to the posts 1202 may be slowly reduced, while gradually increasing the positive pressure subjected to the posts 1204. The posts 1204 may adhere to the external body. The locomotion of the system 100 may be achieved by repeating the simultaneous increase and decrease of the pressure subjected to the consecutive posts.

Moving on, referring to FIG. 13A, the system 100 may be adapted on two arms of a gripper system and the object 1300 may be placed in between the two arms. Referring to FIG. 13B, the arms of the gripper system may be moved towards the object 1300 to establish contact with the object 1300. Referring to FIG. 13C, the system 100 may be inflated (microscale), after the macroscale gripper movement. The inflation may cause the posts 114 to mechanically interlock with the surface of the object 1300, and greatly increase the strength of adhesion. That is to say, the system 100 is subjected to negative or positive pressure only after the posts 114 establish contact with the object 1300.

Although embodiments have been described with reference to specific example embodiments, it will be evident that various modifications and changes may be made to these embodiments without departing from the broader spirit and scope of the system and method described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications, these should not be construed as limiting the scope of the invention but as merely providing illustrations of some of the personally preferred embodiments of this invention. Thus, the scope of the invention should be determined by the appended claims and their legal equivalents rather than by the examples given.

1. A system for controllably establishing adhesion or friction with an external body, the system comprising:
a channel member comprising a plurality of fluidic channels communicating positive or negative pressure; and
an interface member disposed over the channel member, the interface member comprising:
exposed portions that are flexible and exposed to the pressure communicated by the fluidic channels, wherein the exposed portions deform under the pressure communicated by the fluidic channels and reform in the absence of the pressure communicated by the fluidic channels, wherein,
the exposed portions assume a convex shaped configuration when subjected to positive pressure; and
the exposed portions assume a concave shaped configuration when subjected to negative pressure; and
plurality of topographic members provided over the exposed portions, wherein the topographic members move based on the deformation of the exposed portions, wherein the strength of adhesion varies based on the deformation of the exposed portions.

2. The system of claim 1, further comprising a pressure communication member, wherein,
the pressure communication member is provided in between the channel member and the interface member;
the pressure communication member is engaged to the interface member; and
the pressure communication member comprises a plurality of holes aligned with the fluidic channels to communicate the positive or negative pressure to the exposed portions of the interface member.

3. The system of claim 1, wherein the fluidic channels comprises a plurality of independent fluidic channels, wherein pressure communicated by each of the independent fluidic channels is independently controlled.

4. The system of claim 3, wherein, at a given instance, a first set of the independent fluidic channels communicate positive pressure and a second set of the independent fluidic channels communicate negative pressure.

5. The system of claim 3, further comprising:
a plurality of pressure sensors, each of the sensors sensing pressure in one of the independent fluidic channels; and
a controller regulating pressure applied in each of the independent fluidic channels.

6. The system of claim 5, wherein the controller is configured to:
determine change in pressure in at least one of the independent fluidic channels based on input from the respective pressure sensor; and
modify pressure applied to at least one of the remaining independent fluidic channels, if the change in pressure requires compensating of pressure.

7. The system of claim 6, wherein the change in pressure is a result of slipping of one or more of the topographic members, which are provided along the independent fluidic channel in which the change in pressure occurs, from a portion of the external body.

8. The system of claim 1, wherein,
the topographic members are generally perpendicular to the top surface of the interface member when the exposed portions are not subjected to the positive or negative pressure; and
the topographic members are disposed at an oblique angle relative generally to the top surface when the exposed portions are subjected to the positive or negative pressure.

9. The system of claim 1, wherein the topographic members are walls projecting from a top surface of the interface member.

10. The system of claim 1, wherein the topographic members are posts projecting from a top surface of the interface member.

11. The system of claim 10, wherein each of the posts comprises a vertical part and a lateral part extending laterally from the vertical part, wherein the lateral part is configured to interface with the external body.

12. The system of claim 11, wherein each of the posts are tilted by subjecting the exposed portions to negative pressure, to reduce stickiness of the posts against the external body having a smooth surface.

13. The system of claim 10, wherein:
at least a pair of the posts is provided over each of at least the plurality the exposed portions;
each of the movable posts comprises a free end;
the free ends of each of the pair of the posts tilt towards each other when the exposed portion beneath the pair of the posts assumes the concave shaped configuration; and
the free ends of each of the pair of the posts tilt away from each other when the exposed portion beneath the pair of the posts assumes the convex shaped configuration.

14. The system of claim 1, further comprising a robotic gripper comprising multiple arms, each of the arms comprising at least one of the interface member, wherein strength of adhesion, against the external body, of at least one of the interface members is used to manipulate strength of adhesion of at least one of the remaining interface members.

15. The system of claim 1, wherein the positive or negative pressure is communicated by the channel member after at least some of the topographic members have established contact with the external body.

16. The system of claim 1, wherein relative movement is established between the interface member and the external body by altering pressure communicated by the channel members.

17. The system of claim 1 wherein a coefficient of friction of the interface can be actively changed.

18. The system of claim 1, wherein a strength of adhesion to a counterface can be actively changed.

19. The system of claim 18, wherein the strength of adhesion can be optimized through differential pressures being applied, and further comprising a sensing means to sense pressure change within the channels to provide feedback for adhesion optimization.

20. (canceled)

21. The system of claim 1, wherein the topographic members are raised topographic members and mechanically engage with a surface of the external object via its surface roughness features.

22. (canceled)

23. (canceled)
